(19) United States
(12) Patent Application Publication
(10) Pub. No.: US 2005/0009032 A1
(43) Pub. Date: Jan. 13, 2005

(54) Methods and apparatus for characterising cells and treatments

(75) Inventors: Daniel A. Coleman, San Mateo, CA (US); Ge Cong, El Cerrito, CA (US); Aibing Rao, Burlingame, CA (US); Eugeni A. Vaisberg, Foster City, CA (US)

Correspondence Address: BEYER WEAVER & THOMAS LLP, P.O. BOX 778, BERKELEY, CA, 94704-0778, US

(73) Assignee: CYTOKINETICS, INC.

(21) Appl. No.: 10/615,116

(22) Filed: Jul. 7, 2003

(51) Int. Cl. C12Q 1/68; G06K 9/00

(52) U.S. Cl. 435/6; 382/128

(57) ABSTRACT

Methods, data processing apparatus and computer program products for characterising cells and the affect of treatments administered to cells are disclosed. In particular methods of identifying bi-nuclear cells are described which include capturing an image of a plurality of marked cells and processing image to obtain features of the plurality of cells. The features are analyzed to determine whether the feature is indicative of bi-nuclear cells. Those cells for which the first feature is indicative of bi-nuclear cells are identified as being bi-nuclear. Three algorithms in particular are described. A first algorithm can be used to determine the number of nuclei in an image of a nuclear component by determining the number of concave regions within the outline of the image. A second algorithm uses a measure of the amount of cytoplasmic material between a pair of nuclei to identify bi-nuclear cells. A third algorithm uses the statistics of the spatial distribution of objects to identify isolated pairs of nuclei which can be considered to be from the same cell.

### FIELD OF THE INVENTION

The present invention relates to methods, apparatus and computer program products for characterising cells and for use in assessing the effect of treatments on cells. In particular, the invention relates to identifying bi-nucleated cells and assessing the effect of different treatments administered to cells on cellular activities, actions of properties, including promotion, prevention, delay or other inhibition, based on captured images of the treated cells.

### BACKGROUND OF THE INVENTION

A number of methods exist for investigating the effect of a treatment or a potential treatment, such as a drug or pharmaceutical, on an organism. One approach is to investigate how the treatment affects the organism at the cellular level so as to try and determine the mechanism of action by which the treatments affects the organism. One approach to assessing the effects at a cellular level is to capture images of cells that have been subject to a treatment. However, it can be difficult to accurately determine or otherwise quantify the effect of a treatment using captured cell image based techniques owing to the inherent difficulties of capturing and processing visual information. Hence, there is a need for improved algorithms for analyzing image derived data in order to accurately and reliably characterise the effects at a cellular level of a treatment and also the treatment itself.

One area where this would be particularly beneficial is in the area of oncology and cancers. It is believed that tumours are the result of a break down in the normal regulation of cell division, which normally occurs through a process known as the cell cycle. The cell cycle has a number of stages. In eukaryotic cells, the cell cycle generally consists of four stages G₁, S (the DNA synthesis phase), G₂ and mitosis. The stages G₁, S and G₂ are collectively referred to as interphase. During mitosis, the nuclei of eukaryotic cells divide and in parallel, the cytoplasm divides by a process known as cytokinesis. .As a cell leaves G₂, it enters the prophase of mitosis during which the nuclear membrane breaks down and the chromosomes condense. Next metaphase occurs during which the chromosomes are aligned on the equator of the mitotic spindle owing to the action of tubulin containing spindle fibres. Next anaphase occurs during which the daughter chromosomes are pulled toward the poles of the cell by the mitotic spindle. Telophase follows, in which the chromosomes decondense and nuclear membranes form around them and the cell is transiently binuclear. At the same time, a cleavage furrow forms cross the equator of the cell which tightens and eventually divides the cell into two daughter cells and this is cytokinesis.

As cytokinesis is an important part of the cell cycle, it would be advantageous to be able to reliably characterise a cell population in terms of the proportion of cells undergoing cytokinesis (“cytokinetic cells”), or cells in which cytokinesis failed, as this could give a mechanism for robustly investigating the effects of various treatments on the division of cells which could be of use in the drug discovery field or generally in better understanding the interaction between a treatment and cellular operations and activities.

The present invention therefore addresses these issues and provides methods and apparatus for characterising cells, assessing the effects of treatments on cells, and specific algorithms for analysing data derived from images of cells and cell components so as to characterise a cellular property, within a population of cells, based on measures and indications of the existence of bi-nucleated cells.

### SUMMARY OF THE INVENTION

The present invention provides in one aspect, methods, apparatus and software for characterising cellular properties and also for characterising the effects of treatments on cells.

In one aspect of the invention, a method is provided for identifying bi-nuclear cells. A first image of marked cells can be captured. The first image can be processed to obtain a first feature of the cells. The first feature can be analyzed to determine whether the first feature indicates that the cell is a bi-nuclear cell. Those cells for which the first feature is indicative of a bi-nuclear cell can be identified as a bi-nuclear cell.

In another aspect of the invention, a method is provided for assessing the affect of a treatment on a cell. A population of cells can be exposed to the treatment. An image of the cells can be captured. Cellular features can be obtained from the image. The cellular features can be analyzed to assess a property of the cellular feature which is characteristic of bi-nuclear cells. The abundance of bi-nuclear cells can be determined.

In another aspect of the invention, a method is provided for characterising cells. The number of concave portions in the outline of a captured image of a nuclear component of a cell can be determined. The cell can then be characterized based on the number of concave portions.

In another aspect of the invention, a method is provided for identifying bi-nuclear cells. A pair of nuclear components can be identified from a captured image of a nuclear component of cells. A measure of the amount of the cytoplasmic component between the pair of nuclear components can be determined from a captured image of the cytoplasmic component of the cells. The cells can then be characterised based on the amount of the cytoplasmic component.

In another aspect of the invention, a method is provided for identifying pairs of nuclei. A pair of nuclear components can be identified from a captured image of a nuclear component of the cells. A nearest neighbour nuclear component to the pair of nuclear components can be identified. The cells associated with the pair of nuclear components can be characterised based on the separation of the pair of nuclear components and the separation of the next nearest neighbour nuclear component from the pair of nuclear components.

Other aspects of the invention include computer program products and computing devices which can provide the various method aspects of the invention.

These and other features and advantages of the present invention will be described below in more detail with reference to the associated drawings.

### BRIEF DESCRIPTION OF THE DRAWINGS

FIG. 1 is a flow chart depicting at a high level a general image based method for identifying pairs of nuclei so as to assess the effect of a treatment.

FIG. 2 is a flow chart illustrating in greater detail some of the activities carried out during the method illustrated in FIG. 1.

FIG. 3 is a schematic diagram of image capture and data processing apparatus as used during the method illustrated in FIG. 1.

FIG. 4 is a flow chart illustrating some of the image processing operations that can be carried out by the apparatus illustrated in FIG. 3.

FIG. 5 is a flow chart illustrating in greater detail the processes that can be carried out as part of the identification and assessment of the method illustrated in FIG. 1.

FIG. 6 is a process flow chart illustrating an algorithm for assessing nuclear morphology and which can be used to determine the number of nuclei in a cell.

FIG. 7A is a schematic representation of a captured nuclear image illustrating the relationship between the nuclei and the captured image.

FIG. 7B is a schematic representation of a smoothed outline of the nuclear image shown in FIG. 7A illustrating the method illustrated in FIG. 6.

FIGS. 7C, 7D & 7E are respectively schematic representations of a smoothed outline of a nuclear image and the corresponding nuclei illustrating the classification of nuclear objects as part of the method illustrated in FIG. 6.

FIG. 8 is a process flow chart illustrating a nuclear object classification part of the algorithm illustrated in FIG. 6.

FIG. 9 is a high level process flow chart illustrating an algorithm for identifying bi-nuclear cells using inter-nuclear cytoplasmic information.

FIGS. 10A, 10B, 10C and 10D respectively show schematic representations of top and side views of a bi-nuclear cell and two mononuclear cells cell by way of illustration of the general principle underlying the algorithm illustrated in FIG. 9.

FIG. 11 shows a process flow chart illustrating in greater detail the processes involved in the process illustrated in FIG. 9.

FIG. 12 shows a process flow chart illustrating in greater detail a process for determining the amount of cytoplasmic material between a pair of nuclei as used in the process shown in FIG. 11.

FIG. 13A shows a schematic representation of a pair of nuclei illustrating a part of the process illustrated in FIG. 12.

FIG. 13B shows a schematic representation of mapping a line between two nuclei onto cytoplasmic image data illustrating a part of the process illustrated in FIG. 12.

FIG. 14 shows a flow chart illustrating a method of training a classifier part of the process illustrated in FIG. 12.

FIG. 15 shows a plot of a histogram of a population of control cell tubulin image intensity data illustrating the determination of a threshold value as part of the process illustrated in FIG. 14.

FIG. 16 shows a high level process flow chart illustrating an algorithm for identifying pairs of nuclear objects, which can be used to determine the proportion of bi-nuclear cells in a population as part of the method illustrated in FIG. 5.

FIG. 17 shows a schematic representation of three nuclear objects illustrating the processes in the process of FIG. 16 of identifying pairs and isolated pairs of objects.

FIG. 18 shows a process flow chart illustrating in greater detail the process illustrated in FIG. 16.

FIG. 19 is a block diagram of a computer system that can be used to implement various aspects of this invention such as the processes and algorithms illustrated in FIGS. 5, 6, 8, 9, 11, 12, 14, 16 and 18.

### DETAILED DESCRIPTION

Generally, this invention relates to processes and apparatus for use in analysing captured images of cells and components of cells in order to identify bi-nuclear cells, i.e. a single cell having two nuclei. This can occur in cytokinetic cells, i.e. cells undergoing cytokinesis during the cell cycle but whose cytoplasm has not yet divided. The invention can be used to investigate the effect of treatments administered to cells by determining the proportion or number of bi-nuclear cells following a treatment. For example a large number of bi-nuclear cells could be indicative of a treatment that inhibits cytokinesis as otherwise the cytoplasm would divide and cytokinesis would be completed. The failure of cytokinesis would lead to the emergence of a significant number of bi-nuclear cells. However, the methods are not limited to investigating the effect of a treatment administered to the cells on cytokinesis. The methods and apparatus presented in the following can also be used in order to investigate, or otherwise quantify, other cellular behaviour in which bi-nuclear cells can result as will be apparent from the following discussion.

The invention also relates to computer programs, machine-readable media on which is provided instructions, data structures, etc. for performing the processes of the invention. Features of cell components, in particular the nucleus and components of the cytoplasm, which have been derived from captured images of cells are analyzed in order to provide some indication on the extent of occurrence of a biologically relevant phenomenon, such as cytokinesis, the failure of cytokinesis or other phenomena for which bi-nuclear cells are a distinguishing feature. The indication can then be used to help classify or otherwise categorise a treatment that has been applied to the cells.

The general method includes the identification of bi-nuclear cells using images captured by an image capture system. Typically an image will be captured of a cell or plurality of cells, depending on the magnification at which the image is captured and certain markers can be used to highlight in the captured image the component of the cell of interest. The term “marker” or “labelling agent” refers to materials that specifically bind to and label cell components. These markers or labelling agents should be detectable in an image of the relevant cells. Typically, a labelling agent emits a signal whose intensity is related to the concentration of the cell component to which the agent binds. Preferably, the signal intensity is directly proportional to the concentration of the underlying cell component. The location of the signal source (i.e., the position of the marker) should be detectable in an image of the relevant cells.

Preferably, the chosen marker binds indiscriminately with its corresponding cellular component, regardless of location within the cell. Although in other embodiments, the chosen marker may bind to specific subsets of the component of interest (e.g., it binds only to sequences of DNA or regions of a chromosome). The marker should provide a strong contrast to other features in a given image. To this end, the marker should be luminescent, radioactive, fluorescent, etc. Various stains and compounds may serve this purpose. Examples of such compounds include fluorescently labelled antibodies to the cellular component of interest, fluorescent intercalators, and fluorescent lectins. The antibodies may be fluorescently labelled either directly or indirectly.

As part of the general method, the effect of a stimulus or treatment on cells can be investigated using the algorithms described herein. The term “treatment” or “stimulus” refers to something that may influence the biological condition of a cell. Often the term will be synonymous with “agent” or “manipulation.” Stimuli may be materials, radiation (including all manner of electromagnetic and particle radiation), forces (including mechanical (e.g., gravitational), electrical, magnetic, and nuclear), fields, thermal energy, and the like. General examples of materials that may be used as stimuli include organic and inorganic chemical compounds, biological materials such as nucleic acids, carbohydrates, proteins and peptides, lipids, various infectious agents, mixtures of the foregoing, and the like. Other general examples of stimuli include non-ambient temperature, non-ambient pressure, acoustic energy, electromagnetic radiation of all frequencies, the lack of a particular material (e.g., the lack of oxygen as in ischemia), temporal factors, etc.

Specific examples of biological stimuli include exposure to hormones, growth factors, antibodies, or extracellular matrix components. Or exposure to biologics such as infective materials such as viruses that may be naturally occurring viruses or viruses engineered to express exogenous genes at various levels. Biological stimuli could also include delivery of antisense polynucleotides by means such as gene transfection. Stimuli also could include exposure of cells to conditions that promote cell fusion. Specific physical stimuli could include exposing cells to shear stress under different rates of fluid flow, exposure of cells to different temperatures, exposure of cells to vacuum or positive pressure, or exposure of cells to sonication. Another stimulus includes applying centrifugal force. Still other specific stimuli include changes in gravitational force, including sub-gravitation, application of a constant or pulsed electrical current. Still other stimuli include photobleaching, which in some embodiments may include prior addition of a substance that would specifically mark areas to be photobleached by subsequent light exposure. In addition, these types of stimuli may be varied as to time of exposure, or cells could be subjected to multiple stimuli in various combinations and orders of addition. Of course, the type of manipulation used depends upon the application.

As part of the processing of captured images, certain features of the cells can be extract using suitable image processing techniques. The algorithms of the present invention can take this feature data as input in order to carryout their analysis. As used herein, the term “feature” refers to a property of a cell or population of cells derived from cell images and includes the basic “parameters” extracted from a cell image. The basic parameters are typically morphological, concentration, and/or statistical values obtained by analyzing a cell image showing the positions and concentrations of one or more markers bound within the cells. Examples of the various features used by the algorithms are given later on herein. It will be appreciated in the following that some of the algorithms of the present invention can work directly from the feature data, e.g. nuclear position and shape, and do not need to themselves process the images from which the feature data has been obtained, whereas other of the algorithms process image data or use other information contained in an image, together with any required feature data.

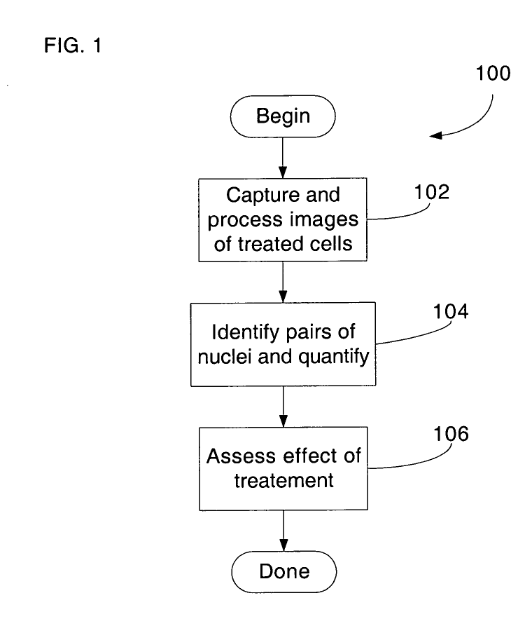

With reference to FIG. 1 there is shown a high level flowchart of a method 100 of investigating the effect of a treatment on cells based on the analysis of captured cellular images. An experiment into the effect of a treatment can typically be carried out by combining sets of assay plates to achieve some scientific purpose. An assay plate is typically a collection of wells arranged in an array with each well holding at least one cell which may have been exposed to a treatment or which provides a control sample. In other embodiments, the experiments are not carried out in multiwell plates. As explained above, a treatment can take many forms and in one embodiment can be a particular drug or any other external stimulus (or a combination of stimuli and/or drugs) to which cells are exposed on an assay plate or have previously been exposed. Experimental protocols for investigating the effect of a treatment will be apparent to a person of skill in the art and can include variations in the dose level, incubation time, cell type and other parameters which are typically varied as part of an experimental protocol. At step 102, images of the treated, marked cells are captured and processed in order to extract the relevant cellular features. As explained above, the cell or components of a cell are marked using a suitable stain or marker which can be detected by an image-capturing device. At step 102 images of the cells and cell parts are captured, stored and processed as will be described in greater detail below.

The cellular features derived from the captured images are then analysed in step 104 in order to identify cells exhibiting the biological phenomenon of relevance. In a preferred embodiment, the cellular features are analysed in order to identify bi-nuclear cells. Some quantitative measure of the extent to which the biological phenomenon is expressed in the cellular population covered by the images can then be determined. The measure can then be used in step 106 to assess the effect of a treatment on the cells. Although the following description will focus on inhibition of cytokinesis, the invention is not limited to assessing the effect of a treatment on cytokinesis alone. The invention can also be applied to investigating the effect of a treatment on the nucleus of cells as a result of other mechanisms of action.

Generally, a wide number of cell components can be detected and analyzed. Cell components can include proteins, protein modifications, genetically manipulated proteins, exogenous proteins, enzymatic activities, nucleic acids, lipids, carbohydrates, organic and inorganic ion concentrations, sub-cellular structures, organelles, plasma membrane, adhesion complex, ion channels, ion pumps, integral membrane proteins, cell surface receptors, G-protein coupled receptors, tyrosine kinase receptors, nuclear membrane receptors, ECM binding complexes, endocytotic machinery, exocytotic machinery, lysosomes, peroxisomes, vacuoles, mitochondria, Golgi apparatus, cytoskeletal filament network, endoplasmic reticulum, nuclei, nuclear DNA, nuclear membrane, proteosome apparatus, chromatin, nucleolus, cytoplasm, cytoplasmic signalling apparatus, microbe specializations and plant specializations.

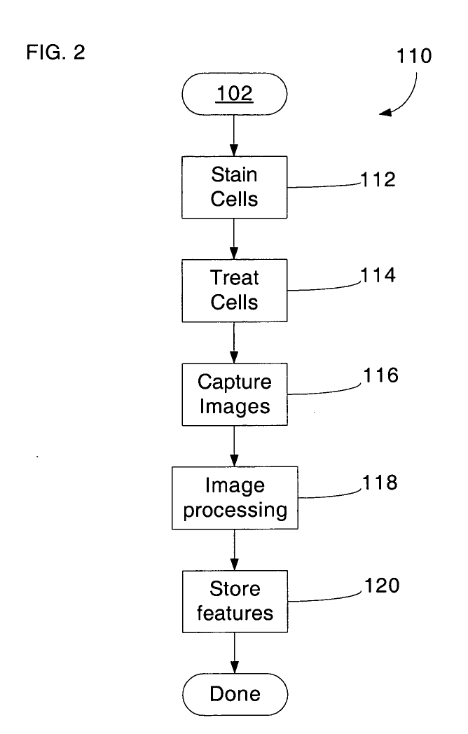

FIG. 2 shows a flowchart 110 illustrating in greater detail some of the operations carried out in step 102 of FIG. 1. In a first step 112, the cells can be stained or otherwise marked so that images can be captured of the cells or cell components of interest. Different cell components can be marked using different stains as is known in the art. At least the nuclei of the cells are stained. Suitable stains for marking the nucleus would include DAPI, Hoechst #33258 and a variety of other stains. A preferred stain would be Hoechst #33258 which provides good contrast for capturing images of nuclear DNA. As well as staining nuclear components, cytoplasmic components of the cell can also be marked with appropriate stains. According to various embodiments of the invention, various different cytoplasmic components can be marked, including Golgi apparatus, cytoskeletal components, the cellular membrane, soluble cytoplasmic proteins, mitochondria, endoplasmic reticulum, endosomes, lysosomes and others. As well as staining the nucleus, the nuclear envelope can also be stained with a suitable marker.

After the cells have been appropriately stained, a treatment 114 can be applied to the cells. A treatment can be of any type which can affect the behaviour of a cell as explained above. The cell may be treated using a chemical agent which can be any type of chemical or chemical compound and may in particular be a potential drug or any other type of therapeutic agent. Typically, a chemical agent may be delivered in a solution and/or with other compounds or treatments, and at varying dose levels. The cells may also be exposed to a biological treatment, such as a virus, protein or by having the cells' DNA modified by any other means by which a biological effect may be exerted on the cells.

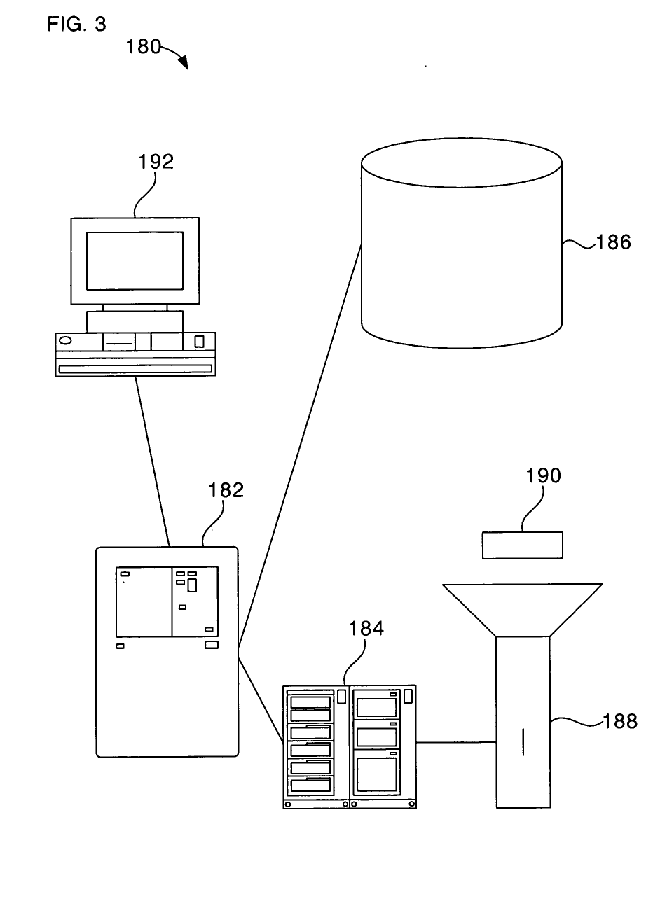

After the cells have been treated, in a next step 116 images of the cells and cellular components are captured using any suitable image capture system. A particular embodiment of a suitable image capture system is shown in FIG. 3 and will be briefly described.

FIG. 3 shows a schematic block diagram of an image capture and processing system which can be used to capture the images of cells or cell parts during step 116. FIG. 3 is a simplified system diagram 180 of an image capture and image processing system. This diagram is merely an example and should not limit the scope of the claims herein. One of ordinary skill in the art would recognize other variations, modifications, and alternatives. The present system 180 includes a variety of elements such as a computing device 182, which is coupled to an image processor 184 and is coupled to a database 186. The image processor receives information from an image capturing device 188, which includes an optical device for magnifying images of cells, such as a microscope. The image processor and image capturing device can collectively be referred to as the imaging system herein. The image capturing device obtains information from a plate 190, which includes a plurality of sites for cells. These cells can be cells that are living, fixed, cell fractions, cells in a tissue, and the like. The computing device 182 retrieves the information, which has been digitized, from the image processing device and stores such information into the database. A user interface device 192, which can be a personal computer, a work station, a network computer, a personal digital assistant, or the like, is coupled to the computing device. In the case of cells treated with a fluorescent marker, a collection of such cells is illuminated with light at an excitation frequency from a suitable light source (not shown). A detector part of the image capturing device is tuned to collect light at an emission frequency. The collected light is used to generate an image, which highlights regions of high marker concentration.

Sometimes corrections must be made to the measured intensity. This is because the absolute magnitude of intensity can vary from image to image due to changes in the staining and/or image acquisition procedure and/or apparatus. Specific optical aberrations can be introduced by various image collection components such as lenses, filters, beam splitters, polarizers, etc. Other sources of variability may be introduced by an excitation light source, a broad band light source for optical microscopy, a detector's detection characteristics, etc. Even different areas of the same image may have different characteristics. For example, some optical elements do not provide a “flat field.” As a result, pixels near the center of the image have their intensities exaggerated in comparison to pixels at the edges of the image. A correction algorithm may be applied to compensate for this effect. Such algorithms can be developed for particular optical systems and parameter sets employed using those imaging systems. One simply needs to know the response of the systems under a given set of acquisition parameters.

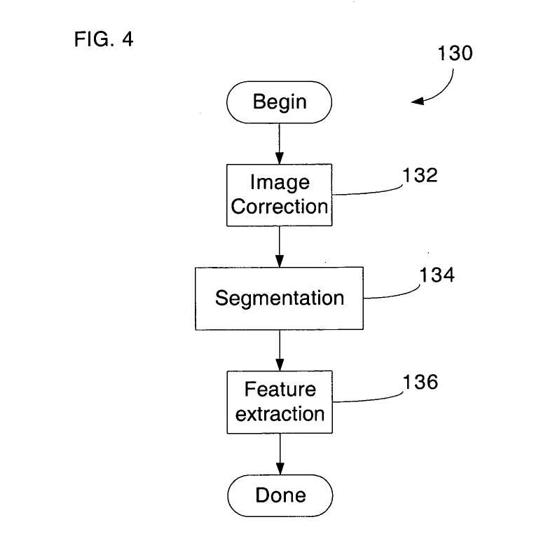

After images of the cells and cell components have been captured 116, the captured images are processed 118 so as to extract cellular features from the images or subsequent analysis. Any suitable image processing steps may be carried out in order to extract relevant cellular features. FIG. 4, which will be discussed further below, illustrates examples of a number of image processing steps that may be carried out during step 118. After the cellular features have been derived from the images, they are stored 120 for future use in database 186 together with any ancillary data relating to the experimental conditions and treatments under which they were obtained.

FIG. 4 shows a flowchart 130 illustrating in greater detail a number of image processing steps carried out and corresponding generally to step 118 of FIG. 2. Not all the steps shown in FIG. 4 are essential. Certain steps may be omitted and other steps may be added depending on the exact nature of the image capture process and markers used. Firstly, the image can be corrected to remove any artefacts introduced by the image capture system and to remove any background or other conventional image correction technique which will improve the quality of the image. Typically, different markers used in an experiment generate radiation at different wavelengths and so either colour images, or separate images for each of the markers may be captured. Therefore different image correction techniques may be used for different markers. Similarly, in the rest of the processes, different techniques may be used, depending on the markers used.

After image correction, a segmentation process 134 is carried out on the images in order to identify individual objects or entities within the image. Any suitable segmentation process may be used in order to obtain nuclear and cellular objects. Typically nuclear DNA markers provide a strong signal and there is a high contrast in the image and an edge detection based segmentation process can be used. For segmenting cells, a watershed type method can be used instead. The segmentation process typically identifies edges where there is a sudden change in intensity of the cells in the image and then looks for closed connected edges in order to identify an object. Segmentation will not be described in greater detail as it is well understood in the art and so as not to obscure the present invention.

Additional operations may be performed prior to, during, or after the imaging operation 116 of FIG. 2. For example, “quality control algorithms” may be employed to discard image data based on, for example, poor exposure, focus failures, foreign objects, and other imaging failures. Generally, problem images can be identified by abnormal intensities and/or spatial measurements.

In a specific embodiment, a correction algorithm may be applied prior to segmentation to correct for changing light conditions, positions of wells, etc. In one example, a noise reduction technique such as median filtering is employed. Then a correction for spatial differences in intensity may be employed. In one example, the spatial correction comprises a separate model for each image (or group of images). These models may be generated by separately summing or averaging all pixel values in the x-direction for each value of y and then separately summing or averaging all pixel values in the y direction for each value of x. In this manner, a parabolic set of correction values is generated for the image or images under consideration. Applying the correction values to the image adjusts for optical system non-linearities, mis-positioning of wells during imaging, etc.

Generally the images used as the starting point for the methods of this invention are obtained from cells that have been specially treated and/or imaged under conditions that contrast the cell's marked components from other cellular components and the background of the image. Typically, the cells are fixed and then treated with a material that binds to the components of interest and shows up in an image (i.e., the marker). Preferably, the chosen agent specifically binds to nuclear DNA, but not to most other cellular biomolecules.

At every combination of dose, cell line, and compound, one or more images can be obtained. As mentioned, these images are used to extract various parameter values of relevance to a biological, phenomenon of interest. Generally a given image of a cell, as represented by one or more markers, can be analyzed to obtain any number of image parameters. These parameters are typically statistical or morphological in nature. The statistical parameters typically pertain to a concentration or intensity distribution or histogram.

Some general parameter types suitable for use with this invention include a cell, or nucleus where appropriate, count, an area, a perimeter, a length, a breadth, a fiber length, a fiber breadth, a shape factor, a elliptical form factor, an inner radius, an outer radius, a mean radius, an equivalent radius, an equivalent sphere volume, an equivalent prolate volume, an equivalent oblate volume, an equivalent sphere surface area, an average intensity, a total intensity, an optical density, a radial dispersion, and a texture difference. These parameters can be average or standard deviation values, or frequency statistics from the descriptors collected across a population of cells. In some embodiments, the parameters include features from different cell portions or cell types.

Examples of some specific cellular and nuclear features and parameters that may be extracted from the captured images during step 136 are included in the following table. Other features and parameters can also be used without departing from the scope of the invention.

| Name of Parameter | Explanation/Comments |
|---|---|
| Count | Number of objects |
| Area | |
| Perimeter | |
| Length | X axis |
| Width | Y axis |
| Shape Factor | Measure of roundness of an object |
| Height | Z axis |
| Radius | |
| Distribution of Brightness | |
| Radius of Dispersion | Measure of how dispersed the marker is from its |
| | centroid |
| Centroid location | x-y position of center of mass |
| Number of holes in closed objects | Derivatives of this measurement might include, for |
| | example, Euler number (=number of objects − number |
| | of holes) |
| Elliptical Fourier Analysis (EFA) | Multiple frequencies that describe the shape of a |
| | closed object |
| Wavelet Analysis | As in EFA, but using wavelet transform |
| Interobject Orientation | Polar Coordinate analysis of relative location |
| Distribution Interobject Distances | Including statistical characteristics |
| Spectral Output | Measures the wavelength spectrum of the reporter |
| | dye. Includes FRET |
| Optical density | Absorbance of light |
| Phase density | Phase shifting of light |
| Reflection interference | Measure of the distance of the cell membrane from |
| | the surface of the substrate |
| 1, 2 and 3 dimensional Fourier | Spatial frequency analysis of non closed objects |
| Analysis | |
| 1, 2 and 3 dimensional Wavelet | Spatial frequency analysis of non closed objects |
| Analysis | |
| Eccentricity | The eccentricity of the ellipse that has the same |
| | second moments as the region. |
| | A measure of object elongation. |
| Long axis/Short Axis Length | Another measure of object elongation. |
| Convex perimeter | Perimeter of the smallest convex polygon |
| | surrounding an object |
| Convex area | Area of the smallest convex polygon surrounding an |
| | object |
| Solidity | Ratio of polygon bounding box area to object area. |
| Extent | proportion of pixels in the bounding box that are |
| | also in the region |
| Granularity | |
| Pattern matching | Significance of similarity to reference pattern |
| Volume measurements | As above, but adding a z axis |
| Number of Nodes | The number of nodes protruding from a closed |
| | object such as a cell; characterizes cell shape |
| End Points | Relative positions of nodes from above |

After the features have been extracted 136 from the image they are stored 120 in database 186, and analysis of the features is carried out in order to assess the effect of the treatment on the cells.

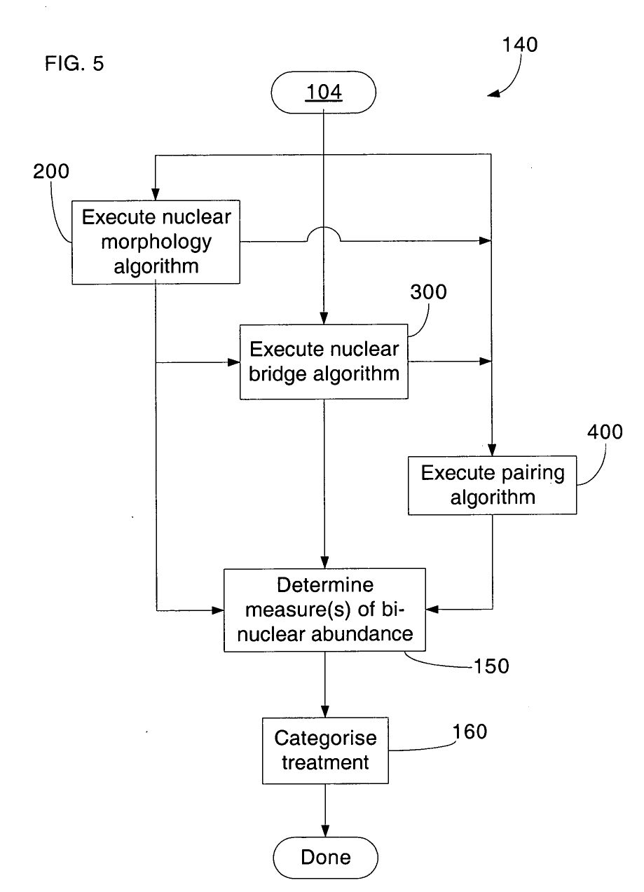

FIG. 5 shows a flow chart 140 illustrating the inter-relationship of three particular algorithms for identifying and quantifying bi-nuclear cells in a cellular population, and corresponds generally to step 104 of FIG. 1. The three particular algorithms for categorising the population of cells in an image will be described in greater detail below. These algorithms may be used separately or in any combination with each other, in order to validate their respective results and improve the categorisation of the treatment based on the analysis of the cellular population.

A first algorithm 200 can be used to characterises the nuclear morphology of individual cells. This algorithm can be used to determine whether a nuclear object in an image can be considered to be a single or multi-nuclear object. Hence this algorithm can be used where only a nuclear stain has been used and helped to categorise the effect of the treatment on the nuclei of cells, e.g. as expressed in the nuclear division immediately prior to cytokinesis. A second algorithm 300 takes into account inter-nuclear properties in order to determine whether a particular cell can be characterised as being bi-nuclear. It is particularly suitable for assessing the effect of a treatment on cytokinesis, or inhibition thereof, in a population of cells. As this algorithm uses information relating to the cytoplasm, a cytoplasmic marker is also used in conjunction with the nuclear marker information so as to try and characterise cells as cytokinetic or not. The inter-nuclear algorithm 300 can be used alone, or subsequent to the nuclear morphology algorithm 200 as will be described in greater detail below. These two algorithms can be used to classify the nuclear status of each cell.

A third pairing algorithm 400 can be used to identify a pairing characteristic of cells within a cellular population. Contrary to the other two algorithms, this algorithm does not determine whether a particular cell is bi-nuclear or not, but rather provides a measure of the number of bi-nuclear cells in a population of cells, without assigning each individual cell to a particular class. In a particular embodiment, the pairing algorithm can identify pairs of nuclear objects which can be likely characterised as corresponding to a cell undergoing cytokinesis. Therefore this algorithm can also give a measure of the proportion of cytokinetic cells in the population. The pairing algorithm can be used alone or can be used in conjunction with either or both of the other algorithms. Preferably, the nuclear morphology algorithm is used in order to identify mono-nucleate objects before carrying out the pairing algorithm to identify likely cytokinetic cells.

After one or more of the algorithms has been carried out, at step 150 some measure or measures of the abundance of bi-nuclear cells in the cellular population is determined. A separate measure can be obtained from each algorithm or the separate measures can be combined to provide a single measure. For example the proportion of cells in the cellular population which are undergoing, failed to, or have recently undergone cytokinesis can be obtained. The measure of bi-nuclear cells, which can provide a measure of the inhibition of cytokinesis (as the greater the number of bi-nuclear cells, the less prevalent cytokinesis), obtained in step 150 is then used in step 160 in order to categorise or otherwise classify the treatment.

The metric obtained in step 150 can be evaluated against control or standard values in order to categorise a treatment. For example a treatment may be categorised as prohibiting cytokinesis, inhibiting cytokinesis or having no significant effect on cytokinesis. The treatment may be carried out by simply comparing the proportion of bi-nuclear cells for the treated sample with the proportion of bi-nuclear cells in a standard or controlled sample. Some statistical measure of the difference between the cytokinesis metric for the treated cells and the same cytokinesis metric evaluated for different treatments and/or control samples may be used in order to provide a confidence in the categorisation of the treatment as having an effect on cytokinesis. Any suitable statistical test may be used, such as Fisher's exact test or a Student T-test. These tests, and other statistical tests, can be used to determine the confidence with which it can be assumed that the treated cells and control cells do come from distinct groups and hence that the treatment has had a genuine effect on the treated cells. Other statistical tests can be used.

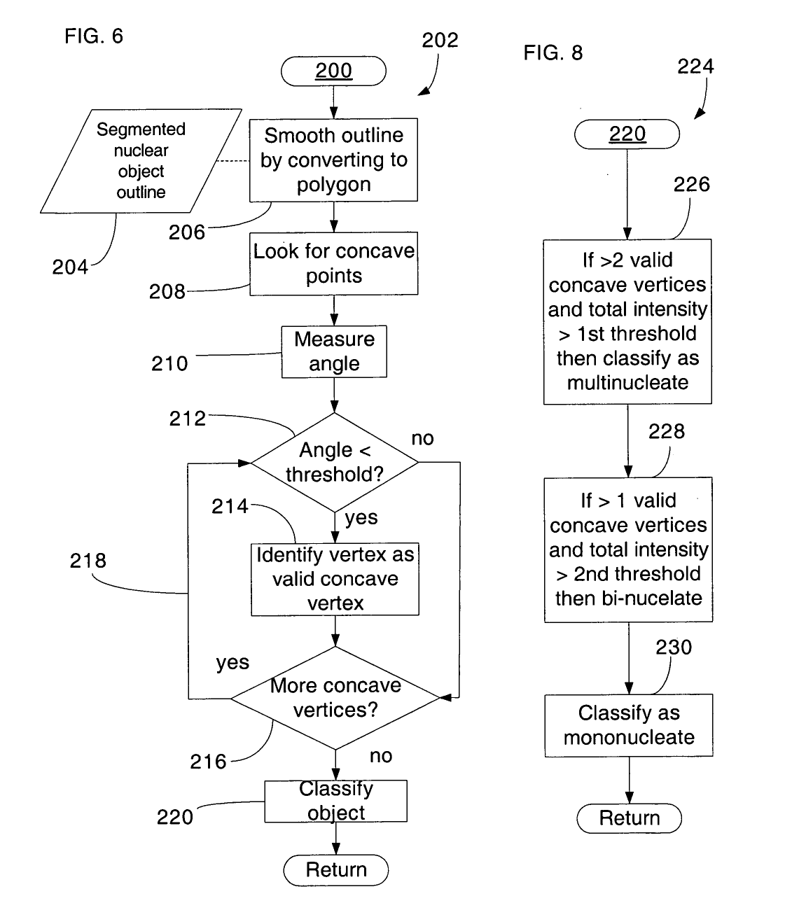

With reference to FIG. 6, there is shown a flow chart 202 illustrating a number of the steps involved in the nuclear morphology algorithm 200. The nuclear morphology algorithm can determine the number of nuclei in a segmented nuclear object obtained from an image of stained nuclear components. In a preferred embodiment, the nuclear components are nuclei. However, other nuclear components which are susceptible to staining could also be used. In one embodiment, the nuclear DNA is marked.

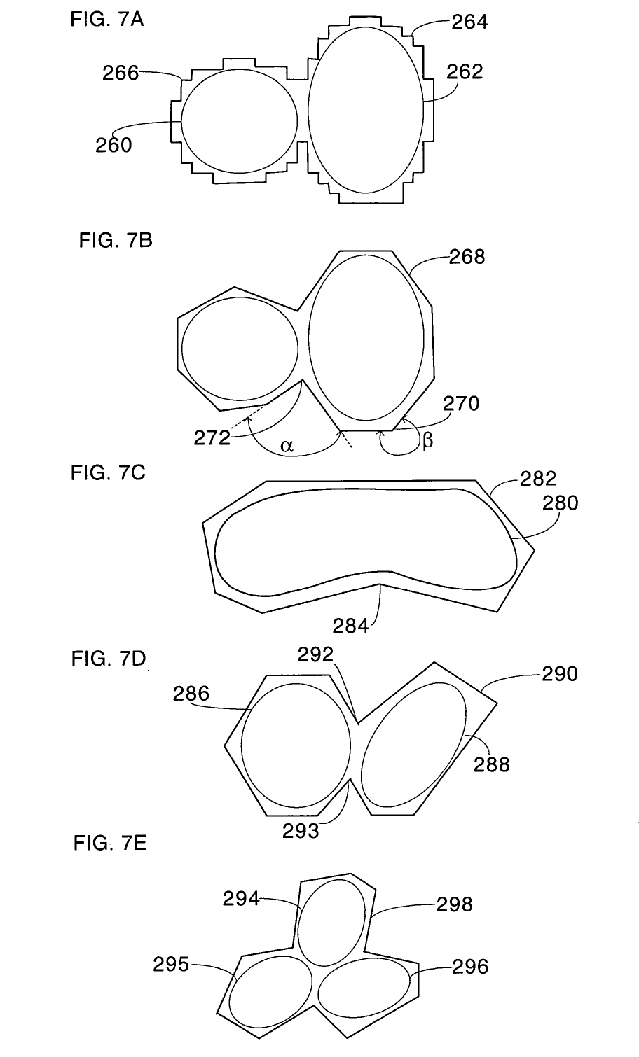

The algorithm 200, takes as input data 204 representing the outline of a single segmented nuclear object 204. As illustrated in FIG. 7A, owing to the resolution of the image capturing device, what may in fact be two separate nuclei 260, 262 may appear as a single nuclear object 264 in a captured image. This will depend on a number of factors, including the resolution of the image capturing device, magnification, the number density of cells in the population and the size of the nuclei. The segmented nuclear object 264 has a perimeter, or outline, 266 which is generally rough owing to pixelation, noise or other artefacts from the image.

In a first step, the algorithm 200 smoothes 206 the outline of the nuclear object so as to remove or reduce the roughness. In a preferred embodiment, the outline is smoothed by converting the outline into an irregular polygon 268 as illustrated in FIG. 7B. In another embodiment, the outline of the polygon can be smoothed by fitting a number of curved segments to the outline of the nuclear object in order to approximate the outline. Polygon 268 in FIG. 7B comprises a number of vertices connected by straight line segments.

At step 208, the algorithm looks for concave regions in the smoothed outline of the nuclear object. In the embodiment illustrated, the concave regions are concave vertices. In one embodiment, the algorithm picks an initial vertex and determines the external angle subtended at that vertex by the adjacent lines of the polygon. For example, at the vertex 270, the external angle is represented by β. As β is greater than 180°, this vertex is not concave, but convex, and so can be discarded for further processing. At vertex 272, the external angle subtended is represented by α. As α is less than 180°, this vertex is a concave vertex and so is retained for further processing. The algorithm evaluates each vertex and measures at step 210 the external angle subtended. If the measured angle of a vertex is 180° or greater, then the vertex can be discarded as not being concave. Those vertices for which the measured angle is less than 180°, are identified as candidate valid concave vertices and are then further evaluated by the algorithm. The algorithm uses the measured angles in order to characterise the candidate valid vertices and the associated region of the object outline as being concave or not.

In a preferred embodiment, a region in the outline of the nuclear object is identified as being concave if the angle subtended by the candidate concave vertex corresponding to that region of the outline falls below a threshold value. As illustrated in greater detail in FIG. 6, for each of the vertices identified as candidate concave vertices, it is determined 212 whether the external angle falls below a threshold value. It will be appreciated that any threshold value which reliably discriminates between concave regions in the outline, so as to be reliably indicative of more than one nucleus, can be used. In a preferred embodiment, the threshold angle is approximately 100□. The threshold used should be less than 180°, and is preferably greater than 90°. Threshold angles in the range of 100-120°, have been found to work reliably. If the angle associated with the candidate concave vertex is less than the threshold, then that candidate concave vertex is 214 as being a valid concave vertex, e.g. vertex 272, indicating that the associated region of the outline can also be considered to be a genuine concave region. If the angle associated with the vertex does not pass the threshold 212 then the candidate concave vertex, e.g. 270, is not identified as being a valid concave vertex.

After a candidate concave vertex has been evaluated, the algorithm determines 216 whether there are any remaining concave candidate vertices in the outline to be evaluated, and if so returns to step 212 where the angle for the next region is evaluated. Processing loops 218 in this way until all the candidate concave vertices have been evaluated.

After the outlines have been evaluated, then all of the nuclear objects are classified at step 220 based on the number of valid concave vertices identified each the object's outline. FIG. 8 shows a flowchart 224 illustrating the steps of the object classification step 220 of the algorithm in greater detail. In general, the number of genuine concave regions identified in the outline of the nuclear object are evaluated in order to determine the number of actual nuclei present in the single image object.

At step 226, a nuclear object in the image is classified as multi-nucleate if its outline has two or more valid concave vertices and if the total intensity of radiation detected for the object exceeds a first threshold. The total intensity of the nuclear object image is proportional to the nuclear DNA present in the actual nuclei. Therefore the total intensity of the nuclear image is compared with a first threshold intensity value to determine whether the amount of DNA present in the actual object is indicative of there being more than two nuclei or not. The total intensity for the nuclear image object is looked up and compared with the first threshold and if the intensity of the nuclear object exceeds the threshold, then this reinforces the belief that the object can be classified as being a multi-nucleate (i.e. more than two nuclei) object. Hence the cell associated with the multi-nuclear object can be classified accordingly as multi-nuclear. Any threshold which allows multi-nuclear objects to be discriminated from bi-nuclear objects can be used. In a preferred embodiment, the threshold is set at 1.9 times the average of the total intensity for all of the nuclear objects in the image.

The nuclear intensity threshold provides a second criterion after the number of valid concave vertices in order to reinforce the classification of the cell and make it more reliable. However, the thresholding step does not have to be used. Further, other properties of the nucleus can be used to provide a secondary criterion by which to discriminate truly multi-nuclear objects . Further more, more than one secondary criterion can be used. Any other feature or property of the nucleus which relates to the likely number of actual nuclei present can be used to provide the secondary check criterion and indeed more than one check criterion can be used. However, the total intensity of a captured image of a nuclear object whose nuclear DNA has been stained is a reliable indicator of the amount of DNA present in the nucleus, and has been found to provide a suitable check criterion.

This scenario is illustrated in FIG. 7E which shows three nuclei 294, 295 and 296 and the smoothed outline 298 rendered by step 206 of the algorithm. The intensity of the nuclear object is checked in step 226 to determine whether there appears to be sufficient nuclear DNA present in the object for it to correspond to three actual nuclei. Hence at step 226 all objects which meet the more than two valid concave vertices and nuclear DNA intensity threshold are classified as being multi-nuclear cells. The remaining objects are then assessed in step 228.

At step 228, for each of the remaining objects, it is determined if the nuclear object has more than one valid concave vertex, and whether the total intensity for the object exceeds a second threshold, different to the first threshold. The second threshold is lower than the first threshold. In a preferred embodiment, the second threshold is approximately 1.1 times the average of the total intensity for all of the nuclear objects in the image. If the object passes both of these criteria, then the nuclear object can be classified as including two actual nuclei and therefore being bi-nucleate, and the associated cell classified accordingly.

FIG. 7D shows two nuclei, 286, 288 and the smoothed outline 290 generated by the algorithm. The vertices 292 and 293 have bother previously been identified as valid concave vertices and the total nuclear DNA intensity is sufficient to pass the second threshold and so this object can be identified as a bi-nuclear object. Again, the use of the second threshold as a second criterion is optional as is the use of other criteria in order to validate the classification of the number of nuclei based on the number of genuine concave regions identified. Hence, during step 228, all of the objects under evaluation meeting the more than one valid concave vertex and the second intensity threshold are classified as bi-nuclear. Those objects not meeting both criteria are then classified in step 230.

The remaining objects are classified in step 230 as being mono-nucleate, i.e. having a single nuclear object. FIG. 7C shows a single nucleus 280 and the smoothed outline 282 rendered by step 206 of method 200. As can be seen, the smooth outline includes a vertex 284 having an angle which subtends less than 180□, however, that vertex did not pass the angle threshold step 212 and so was not passed to step 220 for classification. Hence step 230 classifies those objects which have more than one concave region but failed the 2^{nd} threshold, or which had one or less concave regions, as being mono-nuclear.

Hence as a result of step 220, the physical cell associated with the nuclear object that has been imaged has been classified as being mono, bi or multi nucleate. Hence, cells which have two nuclei close together, identified as bi-nucleate in the algorithm, are likely to be cells which have not undergone cytokinesis and therefore the algorithm helps to identify cytokinetic cells based on the morphology of captured images of nuclear components. However, the algorithm is not limited only to identifying cytokinetic cells, or cells in which cytokinesis has been disrupted, and can be used to identify other biological phenomena in which the number of nuclei associated with a cell or cells can be used as a predictor or indicator of the biological mechanisms occurring.

After all the nuclear object images have been evaluated, the nuclear morphology algorithm is completed at step 224. Hence the nuclear morphology algorithm has identified the nuclear objects in the image and the associated cells in the cell population covered by the image, as being mono-nucleate, cytokinetic or multi-nucleate.

Returning to the general method illustrated in FIG. 5, at step 150, a measure of the proportion of bi-nuclear cells for the cell population can be obtained from the nuclear morphology algorithm alone. A measure of bi-nuclear cell abundance in the population is calculated at step 150. In one embodiment the measure of bi-nuclear cell abundance is the proportion of cells in the image which have been identified as bi-nucleate. For example, X % of the cell population can be identified as being bi-nuclear. At step 160, the treatment to which the cells in the population have been subjected to can then be characterised based on the proportion of bi-nuclear cells.

Characterisation of the treatment can be based on a simple comparison of the proportion of bi-nuclear cells in the treated population with the typical proportion of bi-nuclear cells in a control population. If there has been an increase, then the treatment can be characterised as inhibiting cytokinesis as the cytoplasm of these cells is not dividing even though nuclear division has occurred. If there is no significant difference between the controlled cell population and treated cell population, then the treatment can be categorised as neutral. If there is a decrease, then the treatment may be categorised as promoting cytokinesis. Other categorisations of the treatment are also envisaged.

Further, statistical tests can be used to determine whether the difference between the treated cell population and control population can be considered to be significant or not. For example, a Fisher's exact test or a Student T-test could be applied to the number or proportion of bi-nuclear cells in the treated and control cell populations in order to evaluate whether the determined measure of bi-nuclear cells, and hence the categorisation of the treatment, can be considered to be significant or not.

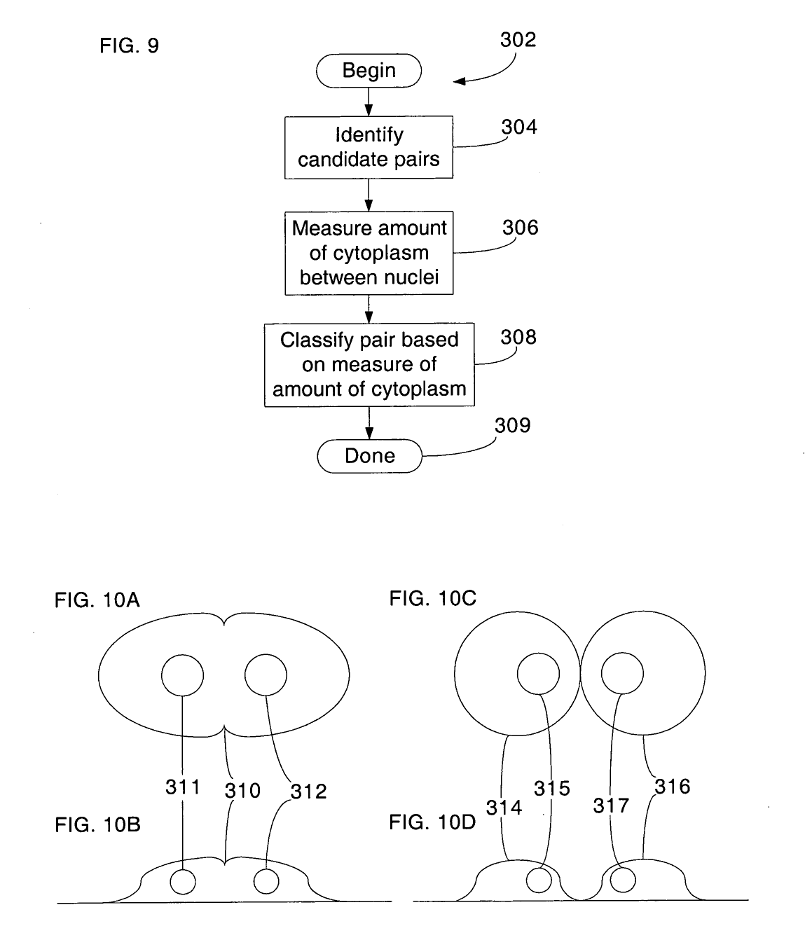

FIG. 9 shows a flow chart 302 illustrating at a high level, the steps involved in an inter-nuclear algorithm 300. This algorithm uses information derived from the cytoplasm of a cell in order to help identify bi-nuclear cells in a cell population from captured images. As both nuclear information and cytoplasmic information are used, this algorithm uses features captured from images of nuclear components and cell cytoplasm components. The principals underlining the algorithm will firstly be described with reference to FIGS. 10A to D.

FIG. 10A shows a plan view of a cell 310 which has failed to undergo cytokinesis and in which the nucleus has split into two daughter nuclei 311, 312 and the cytoplasm has started to divide. FIG. 10B shows a side view along the longitudinal axis of the cell 3101. FIGS. 10A to 10D are schematic and for the purposes of discussion only. FIG. 10C shows a first cell 314 with a nucleus 315 and a second cell 316 with nucleus 317. FIG. 10C shows a plan view and FIG. 10D shows a side elevation of the same cells. These cells are merely nearby or have successfully undergone cytokinesis. As will be apparent from FIGS. 10B and 10D, for cells failing to undergo cytokinesis, or other multi-nuclear cells, there is significantly more cytoplasmic material present between the cell nuclei compared to the situation in which two cells have undergone cytokinesis or are merely adjacent. Algorithm 300 takes advantage of this fact by using a feature derived from a cytoplasmic marker to provide a measure of the proportion of cytoplasmic material between nuclei in order to identify bi-nuclear cells.

In a first step 304, the algorithm 300 identifies candidate pairs of nuclei using segmented nuclear objects for the cellular population. The process then obtains a measure of the amount of cytoplasmic material between the nuclei of the candidate pairs at step 306. A candidate pair is then classified at step 308 depending on whether the measure of cytoplasmic material between the nuclei can be considered to be indicative of a bi-nuclear cell or not. The method completes at step 309. The results of the algorithm can then be fed into step 150 and a measure of bi-nuclear abundance for the cellular population can be calculated.

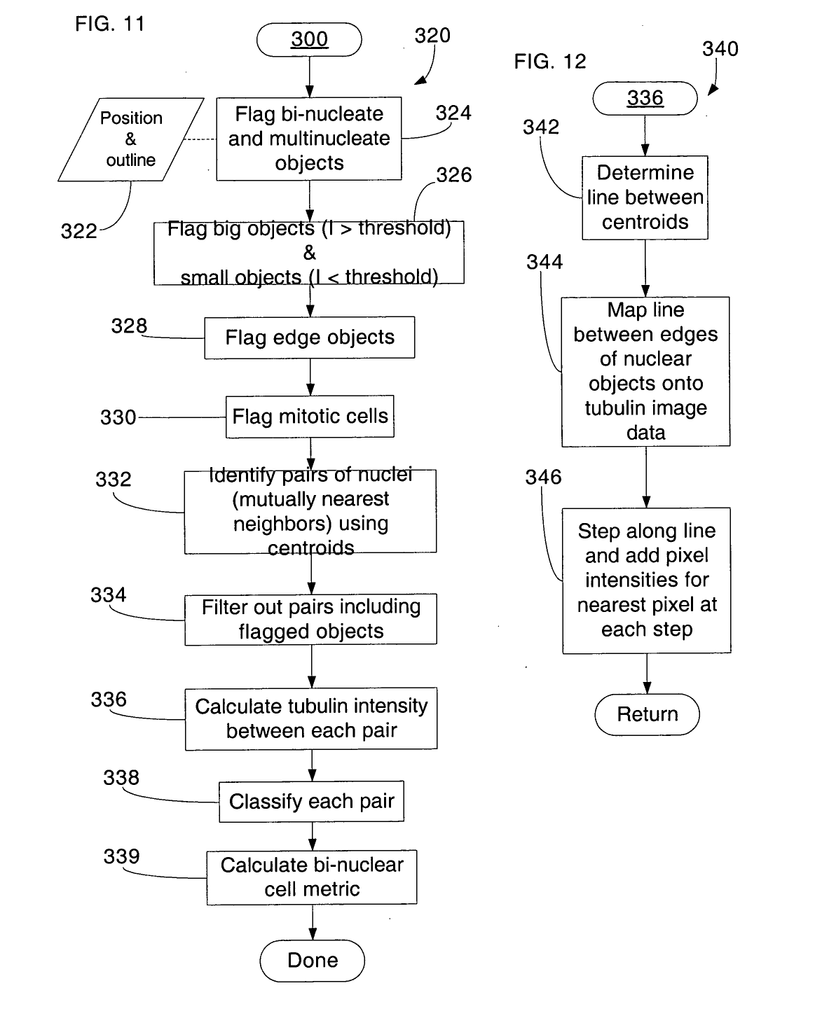

With reference to FIG. 11, there is shown a flow chart 320 illustrating the steps of method 300 in greater detail. The inter-nuclear algorithm receives as input segmented nuclear object position and outline data 322 as extracted from the captured images. A number of optional method steps can be carried out depending on the particular embodiment of the general invention. In an embodiment in which the nuclear morphology algorithm has already been executed for the same image, then nuclear objects which have already been identified as bi- or multi-nucleate are flagged in step 324, however this step is entirely optional. The method may also include an optional step of identifying segmented objects in the image which are considered too big or too small to be genuine nuclear objects (for instance they may be improperly segmented objects). Objects which are considered too big to be nuclear objects can be identified by comparing the intensity for the object with a threshold. In a preferred embodiment, the threshold can be 5,000,000 arbitrary units for object total intensity or 10,000 arbitrary units for object median intensity. Similarly, objects which are considered too small to be genuine nuclei can be flagged by comparing the intensity of the nuclear object image with a second threshold. In one embodiment, the second threshold can be 1,000 arbitrary units for total object intensity or 10 arbitrary units for object median intensity.

At further optional step 328, objects which fall within the edge of the captured image field of view can be flagged so as to remove them from consideration. It is possible that objects falling within the perimeter of the image will not be fully presented in the image and therefore are inaccurate representations of the actual nuclear object. At further optional method step 330, cells which have previously been identified as being mitotic can also be flagged.

At step 332, corresponding generally to step 304, candidate pairs of nuclear objects are identified. For each object, the separation between that object and the remaining nuclear objects in the image is determined based on the centroids of the nuclear objects. Using the separations of the nuclear objects, each nuclear object has its nearest neighbour identified. It is then determined whether the nearest neighbour for that first object and the nearest neighbour object form a mutually nearest neighbour pair. This involves determining whether the first object is also the nearest neighbour of the first object's nearest neighbour. If the pair of objects are mutually nearest neighbours, i.e. the first object is the nearest neighbour of its nearest neighbour, then the pair of nuclei are identified as a candidate pair at step 332. At step 334, the set of candidate pairs identified in step 332 is searched, and those pairs including nuclear objects which have been flagged previously are removed from consideration, e.g. pairs including mitotic cells, edge objects, objects too big or too small or bi- or multi-nuclear objects are removed from further consideration. This helps to identify mutually nearest pairs of apparently mono-nucleate objects which are not undergoing some other cellular process.

As highlighted above, steps 324 to 330 of flagging different types of nuclear objects are optional. Further, step 334 of filtering out unsuitable nuclear objects can be carried out before step 332 of identifying pairs of mutually nearest neighbour nuclear objects. Hence the step of identifying candidate pairs is only carried out on those objects which are believed to be mono-nucleate nuclear objects not undergoing some other biological process. However, it is preferred that filtering of pairs be carried out after all objects have been evaluated to identify mutually nearest neighbour pairs.

At step 336, a measure of the amount of cytoplasm between each mutual nearest neighbour pair of objects is obtained. This step is equivalent to general method step 306. In a particular embodiment, this step is carried out by determining the amount of tubulin present between a pair of nuclei. In particular, the intensity of a captured cellular image of a marker for tubulin is used to calculate or measure the amount of tubulin between the pair of nuclei.

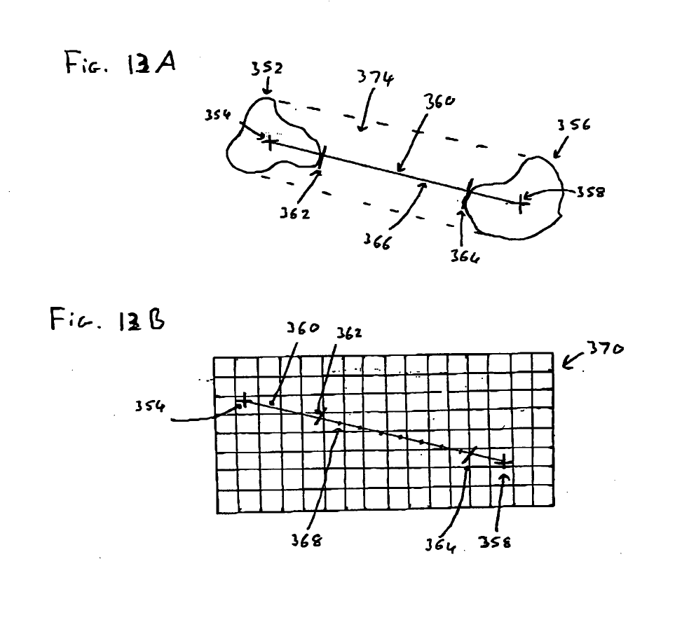

FIG. 12 shows a flow chart 340 illustrating step 336 in greater detail. At step 342, the line between the centroids of a pair of nuclei is determined. This is illustrated schematically in FIG. 13A which shows a first nuclear object 352 having centroid 354 and a second nuclear object 356 having centroid position 358. Line 360 extends between the centroids of the pair of nuclear objects. The edges or outlines of the nuclear objects are used to identify points 362 and 364 on line 360 which are exterior to the nuclei. Therefore portion 366 of line 360 does not extend significantly over nuclear material and should extend mostly over cytoplasm.

At step 344, portion 366 of line 360 extending between the edges of the nuclei is mapped on to image data for the cytoplasmic marker. In a preferred embodiment, the image data is the detected intensity for a tubulin marker. FIG. 13B shows a schematic representation of a set of pixels 370 for a portion of the tubulin image corresponding to the nuclear image and shows the mapping of line 360 from the nuclear image on to the cytoplasmic image data. The tubulin image intensities used are preferably curvature corrected. At step 346, a measure of the amount of tubulin between the nuclei is determined. A number of steps 368 of unit length between points 364 and 362 along line segment 366 are generated. For each point on line segment 366, e.g. 368, the pixel whose position is closest to the point is identified and the tubulin intensity measured for that pixel is added to the sum of tubulin intensity data for all of the points on the line until a measure of the amount of tubulin between the nuclei has been calculated. In another embodiment, instead of using a single line, all those pixels that fall within a band or strip 374 (defined by the shapes of the nuclei) extending between the nuclei are summed to provide the measure of the amount of cytoplasmic material between the nuclei.

Although tubulin has been described above, the invention is not limited to the use of tubulin as a cytoplasmic marker, and other cytoplasmic markers can be used, such as antibodies or fluorescent markers specific to actin, some protein kineses, metabolic enzymes, ATP and other similar cytoplasmic components and structures.

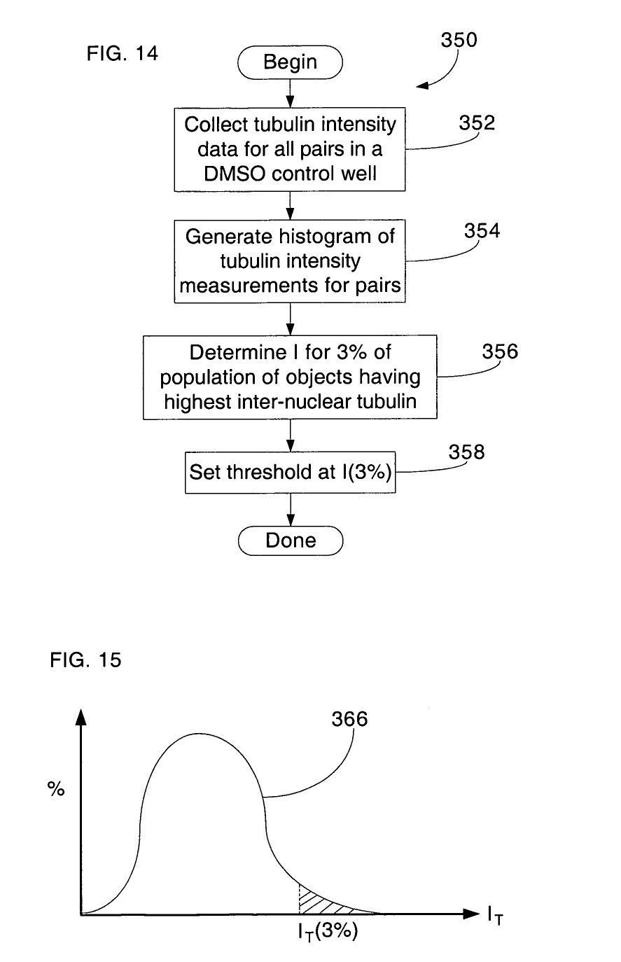

Process flow then returns to the main method and at step 338, each pair of nuclei is classified using the tubulin intensity calculated for each pair. Each pair is classified using a classifier module which has been trained using a control group of cells to identify tubulin threshold intensities against which the calculated tubulin intensity for each pair is compared. FIG. 14 shows a flow chart 350 illustrating the process by which the intensity thresholds used by the classifier can be derived in one embodiment. Either prior to or during an experiment, a set of cells in wells containing DMSO can be provided as control samples. Tubulin intensity data is collected as is nuclear data using different markers. In a similar manner to step 332 of FIG. 11, mutually nearest neighbour pairs of nuclei are identified and the tubulin intensity between each pair is determined using the same process as step 336. This can be carried out for a single well or multiple wells containing the same type of cell as the experimental cells in a control well.

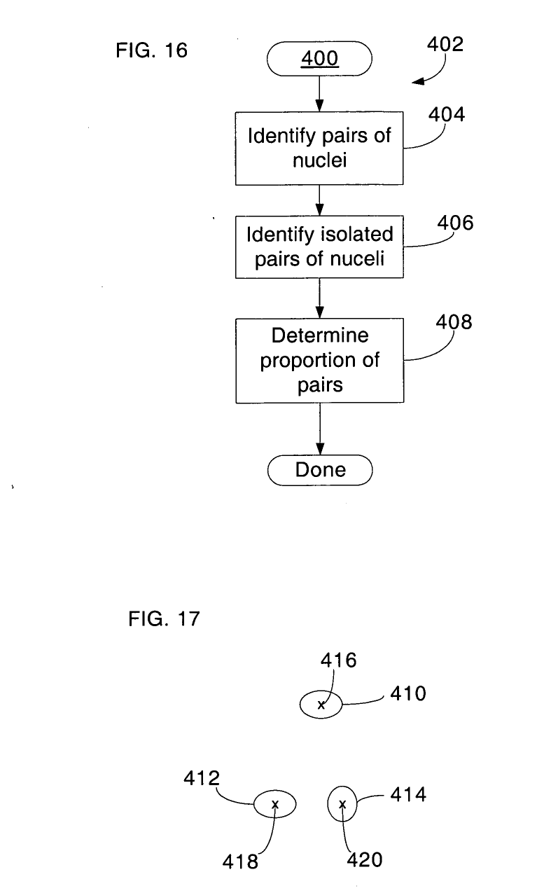

The tubulin intensity data is collected at step 352 and at step 354, data equivalent to a histogram of tubulin intensity measurements for each pair is calculated. It is not necessary to plot a histogram but data indicating the proportion of pairs having a certain tubulin intensity as a function of tubulin intensity (I_{T}) is derived. FIG. 16 shows a plot of a tubulin intensity histogram 366 that can be generated from such data. It has been observed that for a typical control sample, the proportion of cells undergoing cytokinesis, i.e. having two nuclei and a cytoplasm about to divide or dividing, is typically in the range of 4% to 2% of the total cellular population. At step 356, the method determines the intensity (I_{T}(3%)), for a control sample, corresponding to the 3% of the cellular population having the highest measured inter-nuclear tubulin. 3% is a preferred proportion, and in other embodiments, a threshold corresponding to 4% or less of the cellular population or a threshold corresponding to 2% or less of the cellular population can be used.

In greater detail, the percentile corresponding to the intensity threshold to be used can be estimated by assuming a given percentile of the cytokinetic pairs amongst all the image objects in the control cell population. N_{obj} is the number of objects in the image and Nₚₐᵢᵣ is the number of mutually nearest neighbour pairs from the DMSO control well cellular images. For a given object percentile, Q_{obj}, which is assumed to be the proportion of cytokinetic objects, and with Nyo being the number of cytokinetic pairs in the DMSO control wells, then Q_{obj}=N_{cyto}×100/(N_{obj}−N_{cyto}). So that N_{cyto}=(N_{obj}×Q_{obj})/(100+Q_{obj}). Therefore, the estimated percentage of cytokinetic pairs in the training data is Qₚₐᵢᵣ=(N_{cyto}×100)/Nₚₐᵢᵣ. Practically a Q_{obj} of about 3% has been found to provide reliable results so that the pair percentile is set at Q_{DMSO}=100−(N_{obj}×300)/(Nₚₐᵢᵣ×103). The tubulin intensity, I_{T}(3%), corresponding to this percentile for the DMSO training data is then used as the threshold for discriminating between bi-nuclear and non-bi-nuclear pairs of mutually nearest neighbour nuclear objects.

Hence, from the histogram data, the tubulin intensity, I_{T}(3%), corresponding to the 3% of the population having the highest inter-nuclear intensity measurements is obtained and the threshold used in the classifier 338 in the inter-nuclear algorithm 300 is set at this threshold instep 358. The threshold to use can vary between cell types and cell lines, and so cell specific thresholds can be used and similarly the proportion of the cellular population used to identify the threshold value can vary depending on the cell type and cell line.

Returning to step 338, the classifier evaluates each pair of nuclear objects and if the measured tubulin for the pair of objects meets or exceeds the threshold intensity, then the pair of nuclei can be classified as belonging to a bi-nuclear cell as the nuclei are adjacent and the amount of cytoplasmic material between them can be considered sufficiently large to be indicative of the nuclei being present in the same cell and not merely separate adjacent cells.

After each pair in the population has been classified, a bi-nuclear cell abundance metric can be calculated at step 339 to give a measure of the proportion of objects within the cellular population in the image which can be considered to be bi-nuclear cells. One bi-nuclear abundance metric, referred to as a pairing index or metric, that can be used is given by N_{cyto}×100/(N_{obj}−N_{cyto}), where N_{obj} is the number of objects considered and N_{cyto} is the number of cytokinetic/bi-nuclear pairs identified from those same objects.

This pairing metric can be used alone or in combination with the cytokinesis metric obtained from the nuclear morphology algorithm in order to categorise the treatment at step 160.

FIG. 17 shows a flow chart 402 illustrating the pairing algorithm 400 at a high level. The pairing algorithm can be used to identify biologically related pairs of nuclei, e.g. those that are in a cell undergoing cytokinesis or from a cell that has recently undergone cytokinesis. Also this algorithm can be used to identify cells which have not undergone cytokinesis but for which the cells can be considered to be a pair by virtue of the statistical distribution of cells within the population. This can be of use in investigating other aspects of cellular behaviour, such as the effect of a treatment on mobility or other transport property of cells. The preceding two algorithms identifies two objects are deemed a pair. In contrast, the current algorithm identifies individual objects which can be deemed ‘paired’.

The pairing algorithm 400, with reference to FIG. 16, initially identifies pairs of nuclei at step 404. For example, FIG. 17 schematically shows the outlines of three nuclei 410, 412, 414 and their respective centroids 416, 418 and 420. Nuclei 412 and 414 are identified as being a pair of nuclei and at step 406 it is determined whether the pair of nuclei can be considered to be an isolated pair of nuclei. The statistical properties of nearest neighbour distributions for groups of objects are used in order to determine whether nuclei can be considered to be a pair and also whether the pair can be considered to be isolated. Those pairs of nuclei passing both tests are identified as being nuclei from a bi-nuclear cell, and the proportion of bi-nuclear cells for the cellular population is determined at step 408 based on the number of isolated pairs identified.

Expressed in Pseudo Code:

For each object
{

If (nearest neighbour distance<nearest neighbour threshold)
{

object is ‘paired’
if (next nearest neighbour distance>next nearest neighbour
threshold)
{

object is an ‘isolated pair’

}

}

}

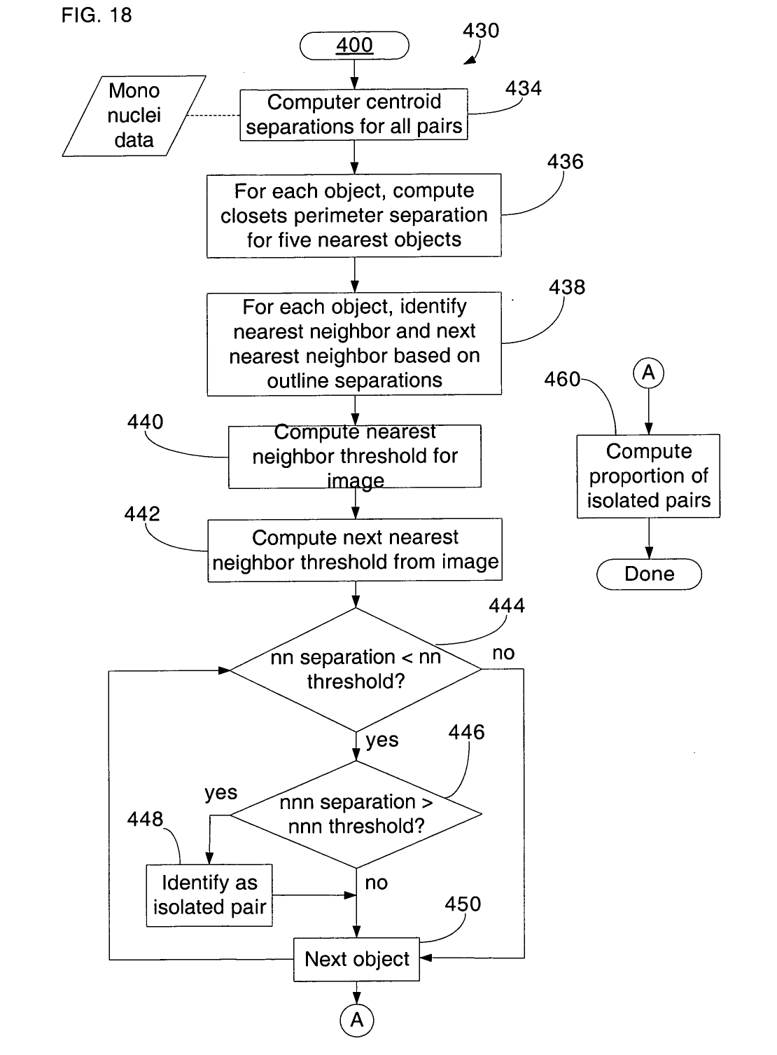

FIG. 18 shows a process flow chart 430 illustrating the pairing algorithm 400 in greater detail. The algorithm takes as input data, the centroid positions and outlines for segmented images of nuclear objects 432. In an embodiment of the overall method, the results of the nuclear morphology algorithm can be used to remove non-mono-nucleate nuclear objects from the image so that the image data used by the pairing algorithm can be considered to relate to single nuclei nuclear objects only. However, it is not essential to use the nuclear morphology algorithm and the pairing algorithm can use nuclear objects that have not been cleaned to remove non-mono-nucleate objects.

At step 434, the separation of the centroids for all the nuclear objects are computed to provide a matrix of pair wise nuclear object separations. At step 436, for each object, the five closest nuclear objects are identified and the separation between the object under consideration and its five nearest neighbours is calculated using the perimeters, or outlines, of the objects, rather than their centroids. It is not essential that the distances be computed between the perimeters and the separation between objects can be computed in other ways. However, using the distance between perimeters has been found to fit the nearest neighbour distributions better than other methods, such as the distance between object centroids. Then at step 438, for each object, and using the perimeter separations, the objects nearest neighbour (nn), e.g. 414 in FIG. 17, and the objects next nearest neighbour (nnn), e.g. object 416 in FIG. 17 are determined. At step 440, a nearest neighbour threshold is computed for the image to identify a nearest neighbour length scale which depends on the density of objects in the image, i.e. the number of objects in the image per unit area. At step 442 a next nearest neighbour threshold is also computed, which similarly depends on the number density of objects in the image. The computation of the nearest neighbour and next nearest neighbour of thresholds will be described in greater detail below.

A nuclear object is then selected for evaluation. At step 444 it is determined if the nearest neighbour separation for the object is less than the nearest neighbour threshold. If not, then the nearest neighbour object is not sufficiently close for the objects to form a pair and so that object can be discarded and a next object is evaluated at step 450. If at step 444 it is determined that the nearest neighbour of an object is sufficiently close for the object to constitute a pair with its nearest neighbour, then the separation of the next nearest neighbour to the object, (e.g. 416 and 412 in FIG. 17) is compared 446 with the next nearest neighbour threshold computed in step 442 and if the next nearest neighbour separation is greater than the threshold, then the pair of objects involved is identified as an isolated pair in step 448. A next object is then evaluated at step 450. If it is determined at step 446 that the next nearest neighbour separation does not exceed the next nearest neighbour threshold, then the pair is not identified as an isolated pair and the next object is evaluated at step 450. Once all the objects have been evaluated, process flow continues to step 460 at which the proportion of isolated pairs is calculated for the cellular population which provides a metric indicative of the number of bi-nuclear cells which can be fed into the treatment categorisation process 160 of the general method.

The calculation of the nearest neighbor (nn) and next nearest neighbor (nnn) thresholds will now be briefly described. The thresholds to use are a function of the number of nuclei in the image. The thresholds are set so that if the nuclei were placed randomly on the image, then we would expect 20% of the nuclei to be classified as paired regardless of the number of nuclei in the image. The following formulae for the thresholds use some results from Spatial Statistics which can be found in Statistics for Spatial Data by Noel Cressie, 1993 published by John Wiley & Sons, Inc. which is incorporated herein by reference for all purposes.

The distribution of nearest neighbors for point objects generated as independent events from a uniform distribution (“complete spatial randomness”) is known as is given by g(w)=2πλw exp(−πλw²) where w is a dummy variable and λ=n/s is the density of objects, where n is the number of objects and s is the size of the image. From this distribution function, the expected proportion of nearest neighbor distances less than a is given by P(nn<a)=1−exp(−πλa²). Hence for a certain proportion of objects, p (e.g. 20% in this example), the nearest neighbor distance aₙₙ corresponding to the proportion of objects p is given by aₙₙ=√−(s/π)log(1−p). Therefore, for a proportion p the nn threshold can be calculated as aₙₙ and is used in step 444.

Using a similar approach, the next nearest neighbor (nnn) threshold is given by aₙₙ=√−(s/πk²)log(1−pk²) which provides the nnn threshold used in step 446.

Each isolated pair can be considered to be a bi-nuclear cell and so the proportion of bi-nuclear cells in the population of cells can be obtained at step 460. As explained above, in step 160, a z-test can be used to compare the proportion of bi-nuclear cells for a treated cell population with the proportion of bi-nuclear cells for a control cell population in order to determine whether the affect of the treatment can be considered to be statistically significant. This can then be used in classifying the treatment, e.g. as inhibiting cytokinesis if there is a statistically relevant large proportion of bi-nuclear cells in the treated cell population.

Generally, embodiments of the present invention employ various processes involving data stored in or transferred through one or more computer systems. Embodiments of the present invention also relate to an apparatus for performing these operations. This apparatus may be specially constructed for the required purposes, or it may be a general-purpose computer selectively activated or reconfigured by a computer program and/or data structure stored in the computer. The processes presented herein are not inherently related to any particular computer or other apparatus. In particular, various general-purpose machines may be used with programs written in accordance with the teachings herein, or it may be more convenient to construct a more specialized apparatus to perform the required method steps. A particular structure for a variety of these machines will appear from the description given below.

In addition, embodiments of the present invention relate to computer readable media or computer program products that include program instructions and/or data (including data structures) for performing various computer-implemented operations. Examples of computer-readable media include, but are not limited to, magnetic media such as hard disks, floppy disks, and magnetic tape; optical media such as CD-ROM disks; magneto-optical media; semiconductor memory devices, and hardware devices that are specially configured to store and perform program instructions, such as read-only memory devices (ROM) and random access memory (RAM). The data and program instructions of this invention may also be embodied on a carrier wave or other transport medium. Examples of program instructions include both machine code, such as produced by a compiler, and files containing higher level code that may be executed by the computer using an interpreter.

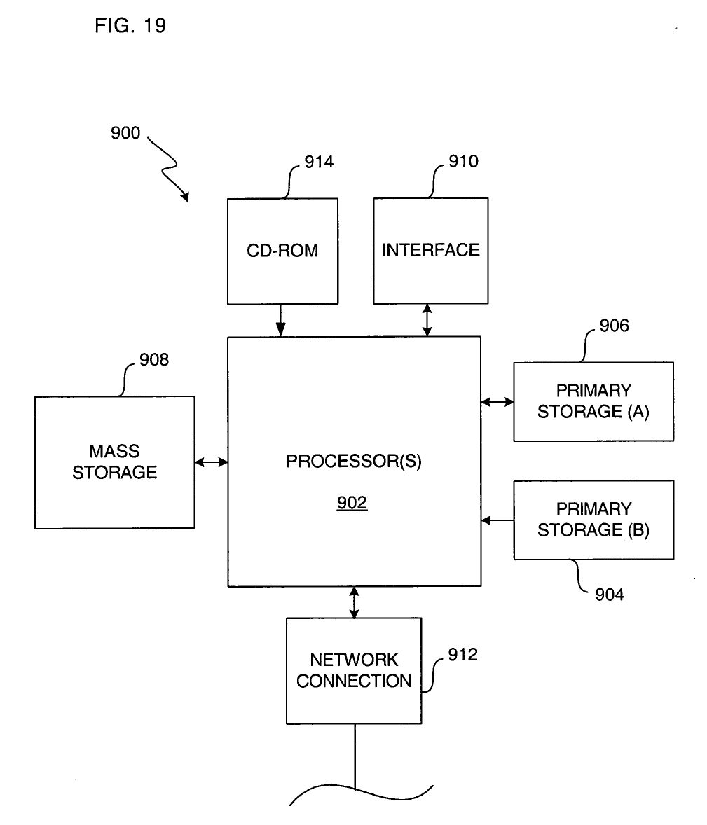

FIG. 19 illustrates a typical computer system that, when appropriately configured or designed, can serve as an image analysis apparatus of this invention. The computer system 500 includes any number of processors 502 (also referred to as central processing units, or CPUs) that are coupled to storage devices including primary storage 506 (typically a random access memory, or RAM), primary storage 504 (typically a read only memory, or ROM). CPU 502 may be of various types including microcontrollers and microprocessors such as programmable devices (e.g., CPLDs and FPGAs) and unprogrammable devices such as gate array ASICs or general purpose microprocessors. As is well known in the art, primary storage 504 acts to transfer data and instructions uni-directionally to the CPU and primary storage 506 is used typically to transfer data and instructions in a bi-directional manner. Both of these primary storage devices may include any suitable computer-readable media such as those described above. A mass storage device 508 is also coupled bi-directionally to CPU 502 and provides additional data storage capacity and may include any of the computer-readable media described above. Mass storage device 508 may be used to store programs, data and the like and is typically a secondary storage medium such as a hard disk. It will be appreciated that the information retained within the mass storage device 508, may, in appropriate cases, be incorporated in standard fashion as part of primary storage 506 as virtual memory. A specific mass storage device such as a CD-ROM 514 may also pass data uni-directionally to the CPU.

CPU 502 is also coupled to an interface 510 that connects to one or more input/output devices such as such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 502 optionally may be coupled to an external device such as a database or a computer or telecommunications network using an external connection as shown generally at 512. With such a connection, it is contemplated that the CPU might receive information from the network, or might output information to the network in the course of performing the method steps described herein.

Although the above has generally described the present invention according to specific processes and apparatus, the present invention has a much broader range of applicability. In particular, aspects of the present invention is not limited to any particular kind of cellular process and can be applied to virtually any cellular process where an understanding of the affect of a treatment on a cell is desired. Thus, in some embodiments, the techniques of the present invention could provide information about many different types or groups of cells, substances, cellular processes and mechanisms of action, and genetic processes of all kinds. One of ordinary skill in the art would recognize other variants, modifications and alternatives in light of the foregoing discussion.

1. A method for identifying bi-nuclear cells, comprising:
capturing at least a first image of a plurality of marked cells;
processing the first image to obtain at least a first feature for each of the plurality of cells;
analyzing the first features for the plurality of cells to determine whether the first feature is indicative of a bi-nuclear cell; and
identifying those cells for which the first feature is indicative of a bi-nuclear cell as being a bi-nuclear cell.

2. The method as claimed in claim 1, in which the first feature is a nuclear feature.

3. The method as claimed in claim 2, in which the first feature is a nuclear morphology.

4. The method as claimed in claim 3, in which analyzing the nuclear morphology further includes determining the number of nuclei present in the first feature.

5. The method as claimed in claim 4, in which analyzing the nuclear morphology includes identifying concave regions in the periphery of the shape of the nuclear feature.

6. The method as claimed in claim 5, in which cells are identified as being bi-nuclear if more than one concave region is identified.

7. The method as claimed in claim 2, in which analysing the first feature further includes analysing the spatial distribution of the first feature.

8. The method as claimed in claim 7, in which analysing the first feature further includes identifying at least one pair of first features.

9. The method as claimed in claim 8, further including: processing the first image to obtain a second feature indicative of a cytoplasmic component; and
wherein analyzing further comprises assessing the cytoplasmic component between the pair of first features.

10. The method as claimed in claim 9, in which identifying further comprises determining whether the amount of the cytoplasmic component exceeds a threshold value.

11. The method as claimed in claim 10, in which the threshold value relates to a control group of cells.

12. The method as claimed in claim 7, and further comprising identifying pairs of nearest neighbour first features.

13. The method as claimed in claim 12, and further comprising identifying the next nearest neighbour first features to a pair of nearest neighbour first features.

14. The method as claimed in claim 13, and further comprising identifying cells as being bi-nuclear when the pair of nearest neighbours are separated by less than a first threshold and the pair of nearest neighbours are separated from the next nearest neighbours by more than a second threshold.

15. A computer program product comprising a machine readable medium on which is provided program instructions for identifying bi-nuclear cells from a captured image of a plurality of marked cells, the instructions comprising:
code for processing the first image to obtain at least a first feature for each of the plurality of cells;
code for analyzing the first features for the plurality of cells to determine whether the first feature is indicative of a bi-nuclear cell; and
code for identifying those cells for which the first feature is indicative of bi-nuclear cells as being bi-nuclear cells.

16. A computing device comprising a memory device configured to store at least temporarily program instructions for identifying bi-nuclear cells from a captured image of a plurality of marked cells, the instructions comprising:
code for processing the first image to obtain at least a first feature for each of the plurality of cells;
code for analyzing the first features for the plurality of cells to determine whether the first feature is indicative of a bi-nuclear cell; and
code for identifying those cells for which the first feature is indicative of a bi-nuclear cell as being bi-nuclear cells.

17. A method for assessing the affect of a treatment on a cell, comprising:
exposing a population of cells to the treatment;
capturing an image of a plurality of cells from the population;
obtaining a plurality of cellular features from the image;
analyzing the plurality of cellular features to assess a property of the cellular feature characteristic of bi-nuclear cells; and
determining the abundance of bi-nuclear cells.

18. A method as claimed in claim 17, and further comprising classifying the treatment based on the abundance of bi-nuclear cells.

19. A method as claimed in claim 17, in which the plurality of cellular features includes nuclear features.

20. A method as claimed in claim 19, in which the plurality of cellular features further includes cytoplasmic features.

21. A method as claimed in claim 18, wherein the treatment is classified in terms of its affect on cytokinesis.

22. A method as claimed in claim 18, further comprising applying a statistical test to the abundance of bi-nuclear cells in the treated cell population and the abundance of bi-nuclear cells in a control population in order to determine the significance of the affect of the treatment on the treated cell population.

23. A method for characterising cells, comprising:
determining, from a captured image of a nuclear component of a plurality of cells, the number of concave portions in the outline of the image of the nuclear component; and
characterising the cell based on the number of concave portions.

24. The method as claimed in claim 23, further comprising smoothing the outline of the image of the nuclear component.

25. The method as claimed in claim 23, further comprising identifying a concave portion in the outline of the image of the nuclear component by determining the angle subtended by adjacent portions of the outline.

26. The method as claimed in claim 25, wherein identifying a concave portion further includes determining whether the angle is less than a threshold angle.

27. The method as claimed in claim 24, wherein smoothing the outline of the image of the nuclear component includes converting the outline into a polygon.

28. The method as claimed in claim 23, wherein the cell is characterised based on the number of concave portions identified and a secondary criterion

29. The method as claimed in claim 28, wherein the secondary criterion is indicative of the amount of nuclear material.

30. The method as claimed in claim 23, wherein the cell is characterised as multi-nuclear if more than two concave portions are identified.

31. The method as claimed in claim 23, wherein characterising the cell further includes assessing a further feature of a nuclear image of the nuclear component

32. The method as claimed in claim 31, wherein the further feature of the image of the nuclear component is the total intensity of the image of the nuclear component.

33. The method as claimed in claim 32, wherein the cell is characterised as multinucleate if there are two or more concave portions and the total intensity exceeds a first threshold.

34. The method as claimed in claim 33, wherein the cell is characterized as bi-nuclear if the cell is not characterised as multi-nuclear and has more than one concave portion and the total intensity exceeds a second threshold which is less than the first threshold.

35. A computer program product comprising a machine readable medium on which is provided program instructions for characterising cells, the instructions comprising:
code for determining, from a captured image of a nuclear component of a plurality of cells, the number of concave portions in the outline of the image of the nuclear component; and
code for characterising the cell based on the number of concave portions.

36. A computing device comprising a memory device configured to store at least temporarily program instructions for characterising cells, the instructions comprising:
code for determining, from a captured image of a nuclear component of a plurality of cells, the number of concave portions in the outline of the image of the nuclear component; and
code for characterising the cell based on the number of concave portions.

37. A method of identifying bi-nuclear cells, comprising:
identifying, from a captured image of a nuclear component of a plurality of cells, at least one pair of nuclear components;
determining, from a captured image of a cytoplasmic component of the plurality of cells, a measure of the amount of the cytoplasmic component interposed between the pair of nuclear components; and
characterising the cells based on the measure of the amount of the cytoplasmic component.

38. The method as claimed in claim 37, wherein the measure is the detected intensity of the image of the cytoplasmic component.

39. The method as claimed in claim 38, further including:
identifying a straight path between the pair of nuclear components; and
determining the amount of the cytoplasmic component that falls under the path.

40. The method as claimed in claim 39, wherein the path extends between the centroids of the pair of nuclear components.

41. The method as claimed in claim 40, wherein the amount of cytoplasmic component is determined by summing over the path extending between the peripheries of the nuclear components.

42. The method as claimed in claim 37, wherein a pair of nuclear components is identified as a pair, if the nuclear components are mutual nearest neighbours.

43. The method as claimed in claim 37, further including removing particular nuclear components from the image prior to identifying pairs.

44. The method as claimed in claim 43, wherein the particular nuclear components are selected from the group comprising: nuclear components of mitotic cells; nuclear components it the edge of the image; multinucleate nuclear components; nuclear components having an image intensity exceeding a threshold; and nuclear components having an image intensity below a threshold.

45. The method as claimed in claim 37, wherein characterising the cells further includes comparing the measure of the amount of the cytoplasmic component with a measure of the amount of the same cytoplasmic component for a control group of cells.

46. The method as claimed in claim 45, wherein the measure of the amount for the control group corresponds to the proportion of bi-nuclear cells expected in the control group.

47. The method as claimed in claim 46, wherein the proportion of bi-nuclear cells expected in the control group is not more than 4%.

48. A computer program product comprising a machine readable medium on which is provided program instructions for identifying bi-nuclear cells, the instructions comprising:
code for identifying, from a captured image of a nuclear component of a plurality of cells, at least one pair of nuclear components;
code for determining, from a captured image of a cytoplasmic component of the plurality of cells, a measure of the amount of the cytoplasmic component interposed between the pair of nuclear components; and
code for characterising the cells based on the measure of the amount of the cytoplasmic component.

49. A computing device comprising a memory device configured to store at least temporarily program instructions for identifying bi-nuclear cells, the instructions comprising:
code for identifying, from a captured image of a nuclear component of a plurality of cells, at least one pair of nuclear components;
code for determining, from a captured image of a cytoplasmic component of the plurality of cells, a measure of the amount of the cytoplasmic component interposed between the pair of nuclear components; and
code for characterising the cells based on the measure of the amount of the cytoplasmic component.

50. A method for identifying biologically relevant pairs of nuclei, comprising:
identifying, from a captured image of a nuclear component of a plurality of cells, at least one pair of nuclear components;
identifying, from the captured image, a nearest neighbour nuclear component to the pair of nuclear components; and
characterising the cells associated with the pair of nuclear components based on the separation of the pair of nuclear components and the separation of the next nearest neighbour nuclear component from the pair of nuclear components.

51. The method as claimed in claim 50, wherein characterising the cell includes determining if the separation of the pair of nuclear components is less than a first threshold and the separation of the next nearest neighbour nuclear component and pair of nuclear components is greater than a second threshold.

52. The method as claimed in claim 51, wherein the second threshold is at least twice the first threshold.

53. The method as claimed in claim 51, wherein the separation between the pair of nuclear components is the shortest distance between the outlines of the nuclear components.

54. The method as claimed in claim 50, further comprising identifying a set of candidate pairs of nuclear components.

55. The method as claimed in claim 54, wherein identifying the set of candidate nuclear components includes determining the separation between the centroids of the nuclear components for each of the candidate pairs.

56. The method as claimed in claim 51 wherein the first and second thresholds are computed based on the density of nuclear components in the captured image.

57. The method as claimed in claim 51, wherein the cell associated with the pair of nuclear components is characterised as bi-nuclear if the separation of the pair of nuclear components is determined to be less than the first threshold and the separation of the next nearest neighbour nuclear component and pair of nuclear components is determined to be greater than the second threshold.

58. The method as claimed in claim 57, further comprising determining the proportion of bi-nuclear cells in the captured image.

59. A computer program product comprising a machine readable medium on which is provided program instructions for identifying biologically relevant pairs of nuclei, the instructions comprising:
(a) code for identifying, from a captured image of a nuclear component of a plurality of cells, at least one pair of nuclear components;
(b) code for identifying, from the captured image, a nearest neighbour nuclear component to the pair of nuclear components; and
(c) code for characterising the cell associated with the pair of nuclear components based on the separation of the pair of nuclear components and the separation of the next nearest neighbour nuclear component from the pair of nuclear components.

60. A computing device comprising a memory device configured to store at least temporarily program instructions for identifying biologically relevant pairs of nuclei, the instructions comprising:
code for identifying, from a captured image of a nuclear component of a plurality of cells, at least one pair of nuclear components;
code for identifying, from the captured image, a nearest neighbour nuclear component to the pair of nuclear components; and
code for characterising the cell associated with the pair of nuclear components based on the separation of the pair of nuclear components and the separation of the next nearest neighbour nuclear component from the pair of nuclear components.
